SEQUENCE LISTING (8 sequences)

SEQ ID NO: 1 - protein, 314 aa, H.sapien
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 2 - protein, 10 aa, Artificial Sequence
  cell adhesion domain
DGRGDSVAYG

SEQ ID NO: 3 - protein, 10 aa, Artificial Sequence
  heparin binding homology domain
RKKRSKKFRR

SEQ ID NO: 4 - protein, 7 aa, Artificial Sequence
  casein kinase II phosphorylation consensus region
SGSSEEK

SEQ ID NO: 5 - protein, 24 aa, Artificial Sequence
  C-terminal heparin binding homology domain
SKEEDKHLKFRISHELDSASSEVN

SEQ ID NO: 6 - protein, 14 aa, Artificial Sequence
  alternatively spliced domain
NAVSSEETNDFKQE

SEQ ID NO: 7 - DNA, 27 bp, Artificial Sequence
  forward primer
aacgccgaccaaggaaaactcactacc

SEQ ID NO: 8 - DNA, 27 bp, Artificial Sequence
  reverse primer
ctccttttaattgacctcagaagatgc

(19) United States
(12) Patent Application Publication
(10) Pub. No.: US 2007/0134229 A1
(43) Pub. Date: Jun. 14, 2007

(54) Non-native constitutively active osteopontin

(76) Inventor: Bin Tian, Irvine, CA (US)

Correspondence Address: EDWARDS LIFESCIENCES CORPORATION, LEGAL DEPARTMENT, ONE EDWARDS WAY, IRVINE, CA, 92614, US

(21) Appl. No.: 11/546,087

(22) Filed: Oct. 10, 2006

Related U.S. Application Data
(60) Provisional application No. 60/725,239, filed on Oct. 11, 2005.

(51) Int. Cl. C07H 21/04; C12P 21/06; A61K 38/48; C12N 9/64

(52) U.S. Cl. 424/94.63; 435/69.1; 435/320.1; 435/226; 435/325; 424/422; 536/23.2

(57) ABSTRACT

Non-native constitutively phosphorylated forms of osteopontin are disclosed. Such osteopontin forms can be obtained, for example, using an expression vector comprising an isolated nucleic acid that encodes an osteopontin peptide, operably linked to a promoter wherein the osteopontin peptide is either constitutively phosphorylated or is capable of being constitutively phosphorylated. Also disclosed are mutant osteopontin peptides that are either constitutively phosphorylated, or are capable of being constitutively phosphorylated. The disclosed osteopontin forms have anti-calcification activity and, therefore, numerous applications. They can be used, for example, to create calcification-resistant tissue, or to treat calcification-related disease.

Priority is herewith claimed under 35 U.S.C. §119(e) from co-pending Provisional Patent Application No. 60/725,239, filed Oct. 11, 2005, entitled “NON-NATIVE CONSTITUTIVELY ACTIVE OSTEOPONTIN”. The disclosure of this Provisional Patent Application is incorporated by reference herein in its entirety.

### BACKGROUND OF THE INVENTION

Ectopic calcification is the inappropriate biomineralization of soft tissues, characterized by the deposition of calcium salts in tissues other than teeth or bone. Ectopic calcification, also termed dystrophic calcification, is characteristic of a number of clinically important diseases such as, for example, atherosclerosis, kidney and renal calculus, arthritis, and the calcification of implanted biomaterial such as prosthetic heart valves (see e.g., U.S. Pat. No. 6,878,168), vascular grafts, LVAD (left ventricular assist devices), contact lenses and a total artificial heart. Ectopic calcifications are typically composed of calcium phosphate salts, including hydroxyapatite, but can also consist of calcium oxalates and octacalcium phosphate as seen in e.g., kidney stones.

Tissues affected by ectopic calcification often show evidence of tissue alteration and/or necrosis. Indeed, ectopic calcification is frequently observed in soft tissues as a result of injury, disease, and aging. However, ectopic calcifications are not only associated with cell death. For example, ectopic calcifications may occur in native aortic valve stenosis or in the Monckeberg's type calcification that is seen in blood vessels from diabetic and uremic patients. Although most soft tissues can undergo calcification, skin, kidney, tendons, and cardiovascular tissues appear particularly prone to developing this pathology. In addition, a number of prosthetic devices such as artificial heart valves, are prone to ectopic calcification.

Ectopic calcification can lead to clinical symptoms when it occurs in cardiovascular tissues, particularly when it occurs in arteries and heart valves. In arteries, calcification is correlated with atherosclerotic plaque burden and increased risk of myocardial infarction, increased ischemic episodes in peripheral vascular disease, and increased risk of dissection following angioplasty. In the heart, valves are particularly prone to calcification. Indeed, calcific aortic stenosis is a rather common condition, occurring in approximately 1-2% of the elderly population. Calcific aortic stenosis is characterized by stiffening, tearing, and mechanical failure of the valve. Common conditions such as congenital anomalies, rheumatic fever, inflammatory changes, renal disease, and age are all risk factors for calcific aortic valve stenosis.

Treatment for severe symptomatic calcific aortic stenosis is aortic valve replacement. Indeed, more than 40,000 patients undergo valve replacement each year in the United States alone (see e.g., O'Keefe, J. H., et al. (1991) Postgrad. Med. 89:143). Although valve replacement has resulted in dramatic improvement in longevity and symptoms of patients with valve disease, an important cause for the failure of artificial heart valves is, in fact, calcification of the prosthetic valve.

Unfortunately, current therapies to inhibit calcification of vascular tissues or implants are of limited efficacy. Thus, there is a need in the art for efficacious therapies to treat the conditions associated with ectopic calcification, and thereby provide the necessary relief from pathology and disease.

Molecular medicine holds great promise for delivering new therapies that can overcome the limitations of present day treatments for disease. One candidate molecule for the treatment of ectopic calcifications is osteopontin (OPN), a secreted phosphoprotein that is associated with both normal and pathological mineral deposits.

OPN is normally found in bone, teeth, kidney, and the epithelial lining tissues (Oldberg et al. (1986) J. Biol. Chem. 263:19433-19436; Giachelli et al. (1991) Biochem. Biophys. Res. Commun. 177: 867-873). It is expressed at high levels in bony structures of the body, but also in many tissues, the expression of OPN is increased under conditions of injury and disease. Thus, OPN closely associates with calcified deposits both in normal bone and also in the pathologies of ectopic calcification (see e.g., Agrawal D et al. (2002) J. Natl. Cancer Inst. 94:513-21).

OPN is a multifunctional protein which is involved in bone mineralization, cell adhesion, cell migration, chronic inflammatory disease, and transformation. Osteopontin appears to play roles in both the promotion of calcification and mineralization, and in the inhibition of calcification. It is found at high levels in calcified vascular tissues, but in contrast is also an inhibitor of mineralization of bovine aortic smooth muscle cells in vitro (see e.g., Ohri, R. et al. (2005) Calcif Tissue Int. Epub ahead of print) and is an inhibitor of ectopic calcification in vivo (see e.g., Steitz S. A. et al. (2002) American Journal of Pathology 161:2035-2046).

Osteopontin comprises several structural domains, some of which include an integrin-binding (RGD) adhesive domain (Arg-Gly-Asp sequence), and aspartic acid rich calcium binding regions. OPN is subject to numerous post translational modifications, including: thrombin cleavage, sulfation, glycosylation, trans-glutamination, and phosphorylation.

The phosphorylation and dephosphorylation of proteins play a major role in the regulation of many biochemical processes. The phosphorylation and dephosphorylation of OPN is apparently an important regulatory mechanism for this protein, particularly with regard to its role in ossification processes. Indeed, depending on the phosphorylation state of the protein, OPN can either facilitate (see, e.g., U.S. Pat. No. 6,509,026, and Jono, S. et al. (2000) JBC 275:20197) or inhibit (see, e.g., U.S. Pat. No. 6,551,990) ossification.

Clearly, if the phosphorylation state of the OPN peptide could be controlled, one would gain the ability to control the degree and direction of the ossification processes. Needless to say such control would be of great benefit to those individuals suffering from the various pathologies of ectopic calcification.

Thus, there exists a need to modify osteopontin peptides in a controlled manner so as to direct the activity of the peptides and thereby control the ossification processes. As will be clear from the following disclosure, the present invention provides for this and other needs.

### SUMMARY OF THE INVENTION

The present invention provides for the first time constitutively phosphorylated forms of osteopontin.

In one exemplary embodiment, the invention provides an expression vector comprising an isolated nucleic acid that encodes an osteopontin peptide, operably linked to a promoter wherein the osteopontin peptide is either constitutively phosphorylated or is capable of being constitutively phosphorylated. In another embodiment, the invention provides a cell comprising an isolated nucleic acid that encodes an osteopontin peptide that is either constitutively phosphorylated, or is capable of being constitutively phosphorylated. And in still other exemplary embodiments, the invention provides mutant osteopontin peptides that are either constitutively phosphorylated, or are capable of being constitutively phosphorylated.

In another exemplary embodiment, the present invention provides an isolated nucleic acid encoding forms of human osteopontin that are, or which can be, constitutively phosphorylated.

Other features, objects and advantages of the invention will be apparent from the detailed description which follows.

### DETAILED DESCRIPTION OF THE INVENTION

Definitions

As used herein, the terms “osteopontin” or “osteopontin peptide,” refer to a form of osteopontin or a fragment thereof, capable of performing its intended function in vivo, or in vitro e.g., and possessing anti-calcification activity. Examples of osteopontin peptides useful in the invention include, but are not limited to a constitutively phosphorylated osteopontin, e.g., an osteopontin having anti-calcification activity.

The term “osteopontin” or “osteopontin peptide,” also refers to a recombinant osteopontin, e.g., a human or murine recombinant osteopontin, e.g., the osteopontin secreted from murine B3H cells, and a naturally occurring osteopontin, e.g., the naturally occurring human osteopontin secreted from human osteoblast cells. A full length amino acid sequence of human osteopontin, according to Kiefer M C et al. (1989) Nucleic Acids Research 17(8): 3306, is shown below.

(SEQ ID NO:1)

MRIAVICFCLLGITCAIPVKQADSGSSEEKQLYNKYPDAVATWLNPDPSQ
KQNLLAPQNAVSSEETNDFKQETLPSKSNESHDHMDDMDDEDDDDHVDSQ
DSIDSNDSDDVDDTDDSHQSDESHHSDESDELVTDFPTDLPATEVFTPVV
PTVDTYDGRGDSVVYGLRSKSKKFRRPDIQYPDATDEDITSHMESEELNG
AYKAIPVAQDLNAPSDWDSRGKDSYETSQLDDQSAETHSHKQSRLYKRKA
NDESNEHSDVIDSQELSKVSREFHSHEFHSHEDMLVVDPKSKEEDKHLKF
RISHELDSASSEVN

The term “osteopontin” or “osteopontin peptide,” also refers to mutant osteopontin peptides whose encoding nucleic acid specifically or selectively hybridizes under moderately stringent conditions to a nucleic acid molecule encoding a peptide that corresponds to SEQ ID NO:1.

The term “calcification” refers to a process whereby a tissue or non-cellular material in the body, becomes hardened as a result of the deposition of insoluble salts of calcium, such as calcium phosphate or calcium carbonate, or sometimes insoluble salts of magnesium.

The term “anti-calcification activity” refers to the inhibition, prevention or amelioration of the ectopic calcification of tissues in response to an injury, pathology, or condition, or the inhibition, prevention or amelioration of ectopic calcification on or around an implanted prosthetic device. “Anti-calcification activity” includes preventing calcification from occurring in an individual who may be predisposed to developing ectopic calcification, but who does not yet experience or exhibit symptoms of ectopic calcification (prophylactic treatment), or inhibiting ectopic calcification (slowing or arresting further deposition of calcium salts) and/or causing regression or resorption of the calcified deposits.

The term “constitutively phosphorylated” refers to peptide which, under most physiological conditions, most of the time, possesses to any degree, the activity typically associated with that of the wild type peptide in its phosphorylated state e.g., possesses anti-calcification activity. In an exemplary embodiment, a “constitutively phosphorylated” osteopontin is phosphorylated at particular amino acid residues such that it has the activity of a phosphorylated wild-type osteopontin, e.g., has anti-calcification activity. Within the human osteopontin sequence, there are as many as 29 serine and 2 threonine residues that can be phosphorylated. The phosphorylation of a number of these amino acid residues can cause the activation of osteopontin via a change in the conformation of the protein. In one exemplary embodiment, a constitutively phosphorylated osteopontin is phosphorylated at one or more of the following amino acids selected from the group consisting of serine 26, 27, 62, 63, 76, 78, 105, 108, 120, 126, 129, 191, 234, 280, 291, 308 and threonine 185. The amino acid positions are numbered according to the osteopontin sequence as provided in SEQ ID NO: 1.

A “constitutively phosphorylated” peptide may or may not be phosphorylated to the same extent and/or with the same pattern as the wild type peptide, and may or may not have the same amino acid sequence as the wild type peptide. Indeed, to be a “constitutively phosphorylated” osteopontin peptide of the invention, the peptide need only possess an exhibit, in any degree, e.g., less, the same, or more, of the activity typically associated with that of the wild type peptide in its phosphorylated state e.g., exhibits anti-calcification activity. In an exemplary embodiment, a “constitutively phosphorylated” osteopontin peptide is phosphorylated at a certain subset of amino acid residues such that the peptide exhibits, in any degree, activity typically associated with that of a completely phosphorylated wild type osteopontin e.g., exhibits anti-calcification activity. In an alternative embodiment, a “constitutively phosphorylated” osteopontin peptide is not phosphorylated at all, but rather has a mutant form that permits the peptide to exhibit, in any degree, activity typically associated with that of a phosphorylated wild type osteopontin peptide e.g., has anti-calcification activity, even though the mutant peptide is not itself phosphorylated.

The term “calcification related disease” refers to any condition involving the inappropriate biomineralization of soft tissues. For example, calcification related diseases include but are not limited to the ectopic calcification of soft tissues that occurs in association with diseases such as atherosclerosis, arthritis, and/or vascular grafts. A calcification related disease also includes, but is not limited to the calcification of implanted biomaterial such as prosthetic heart valves, LVAD (left ventricular assist devices), contact lenses and/or a total artificial heart. Calcification related diseases also include genetic disorders such as Fibrodysplasia ossificans progressiva, wherein an individual's muscle progressively turns to bone.

Calcification related diseases are characterized by the deposition of calcium salts in tissues other than teeth or bone. Ectopic calcifications are typically composed of calcium phosphate salts, including hydroxyapatite, but can also consist of calcium oxalates and octacalcium phosphate as seen in e.g., kidney stones.

The term “operably linked” refers to a functional linkage between two or more peptides or nucleic acids. For example, a recombinant nucleic acid is typically comprised of one or more operably linked nucleic acids wherein for example, a nucleic acid expression control sequence (such as a promoter, or array of transcription factor binding sites) is “operably linked” to a second nucleic acid sequence, and the expression control sequence directs transcription of the nucleic acid corresponding to the second sequence. Operable linkage also occurs when two or more peptides are linked to form a “fusion protein”. For example, the functional domains of a fusion protein, comprised of the functional domains from different proteins that have been linked to produce a new peptide with functional properties of the constituent domains, are also said to be “operably linked”.

The terms “nucleic acid” or “polynucleotide” or “nucleic acid sequences” refer to deoxyribonucleotides or ribonucleotides and polymers thereof in either single- or double-stranded form. The term encompasses nucleic acids containing known nucleotide analogs or modified backbone residues or linkages, which are synthetic, naturally occurring, or non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). The term “nucleic acid sequences” encompasses sequences which are obtained or purified from natural sources, as well as sequences which are obtained or constructed from recombinant or synthetic chemical processes.

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nuc. Acid Res. 195081 (1991); Ohtsuka et al., J. Biol. Chem. 260:2605-2608 (1985); Rossolini et al., (1994) Mol. Cell. Probes 8:91-98). The term nucleic acid is used interchangeably with gene, cDNA, mRNA, oligonucleotide, and polynucleotide.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. An example of potassium channel splice variants is discussed in Leicher, et al., (1998) J. Biol. Chem. 273:35095-35101.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The terms “identical” or percent “identity,” in the context of two or more nucleic acids or polypeptide sequences, refer to two or more sequences or subsequences that are the same or have a specified percentage of amino acid residues or nucleotides that are the same (i.e., 80% identity, preferably 85%, 90%, or 95% identity over a specified region), when compared and aligned for maximum correspondence over a comparison window, or designated region as measured using for example one of many sequence comparison algorithms known in the art, or by manual alignment and visual inspection. Such sequences are then said to be “substantially identical.” This definition also refers to the compliment of a test sequence. In one embodiment, the identity exists over a region that is at least about 25 amino acids or nucleotides in length, and in another embodiment, over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art.

An exemplary algorithm suitable for determining percent sequence identity and sequence similarity are the BLAST and BLAST 2.0 algorithms, which are described in Altschul et al. (1977) Nuc. Acids Res. 25:3389-3402 and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin & Altschul (1993) Proc. Nat'l. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For high stringency hybridization, a positive signal is at least two times background, preferably 10 times background hybridization. Exemplary high stringency or stringent hybridization conditions include: 50% formamide, 5×SSC and 1% SDS incubated at 42° C. or 5×SSC and 1% SDS incubated at 65° C., with a wash in 0.2×SSC and 0.1% SDS at 65° C.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C. and a wash in 1×SSC at 45° C. A positive hybridization is at least twice background. Those of ordinary skill will readily recognize that alternative hybridization and wash conditions can be utilized to provide conditions of similar stringency.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to osteopontin, encoded in SEQ ID NO:1, splice variants, or portions thereof, can be selected to obtain only those polyclonal antibodies that are specifically immunoreactive with osteopontin and not with other proteins, except for polymorphic variants, orthologs, and alleles of osteopontin. This selection may be achieved by subtracting out antibodies that cross-react with certain osteopontin orthologs. A variety of immunoassay formats may be used to select antibodies specifically immunoreactive with a particular protein. For example, solid-phase ELISA immunoassays are routinely used to select antibodies specifically immunoreactive with a protein (see, e.g. Harlow & Lane, Antibodies, A Laboratory Manual (1988) for a description of immunoassay formats and conditions that can be used to determine specific immunoreactivity). Typically a specific or selective reaction will be at least twice background signal or noise and more typically more than 10 to 100 times background.

The phrase “selectively associates with” refers to the ability of a nucleic acid to “selectively hybridize” with another as defined above, or the ability of an antibody to “selectively (or specifically) bind to a protein, as defined above.

By “host cell” is meant a cell that contains an expression vector and supports the replication or expression of the expression vector. Host cells may be prokaryotic cells such as E. coli, or eukaryotic cells such as yeast, insect, amphibian, or mammalian cells such as CHO, HeLa and the like, e.g., cultured cells, explants, and cells in vivo.

“Biological sample” as used herein is a sample of biological tissue or fluid that contains osteopontin or nucleic acid encoding osteopontin protein. Such samples include, but are not limited to, tissue isolated from humans. Biological samples may also include sections of tissues such as frozen sections taken for histologic purposes. A biological sample is typically obtained from a eukaryotic organism, in some embodiments, the biological sample is from eukaryotes such as fungi, plants, insects, protozoa, birds, fish, reptiles, and/or a mammal e.g., rat, mouse, cow, dog, guinea pig, or rabbit. In other embodiments, the biological sample is from a primate e.g., a chimpanzee and/or humans.

The term “isolated” refers to a material that is substantially or essentially free from components, which are used to produce the material. For compositions of the invention, the term “isolated” refers to material that is substantially or essentially free from components, which normally accompany the material in the mixture used to prepare the composition. “Isolated” and “pure” are used interchangeably. Typically, isolated components of the invention have a level of purity preferably expressed as a range. The lower end of the range of purity for the component is about 60%, about 70% or about 80% and the upper end of the range of purity is about 70%, about 80%, about 90% or more than about 90%.

As used herein, “pharmaceutically acceptable carrier” includes any material, which when combined with the conjugate retains the conjugates' activity and is non-reactive with the subject's immune systems. Examples include, but are not limited to, any of the standard pharmaceutical carriers such as a phosphate buffered saline solution, water, emulsions such as oil/water emulsion, and various types of wetting agents. Other carriers may also include sterile solutions, tablets including coated tablets and capsules. Typically such carriers contain excipients such as starch, milk, sugar, certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well known conventional methods.

As used herein, “administering” means oral administration, administration as a suppository, topical contact, intravenous, intraperitoneal, intramuscular, intralesional, or subcutaneous administration, administration by inhalation, or the implantation of a slow-release device, e.g., a mini-osmotic pump, to the subject. Administration is by any route including parenteral and transmucosal (e.g., oral, nasal, vaginal, rectal, or transdermal), particularly by inhalation. Parenteral administration includes, e.g., intravenous, intramuscular, intra-arteriole, intradermal, subcutaneous, intraperitoneal, intraventricular, and intracranial. Moreover, where injection is used to treat a tumor, e.g., induce apoptosis, administration may be directly to the tumor and/or into tissues surrounding the tumor. Other modes of delivery include, but are not limited to, the use of liposomal formulations, intravenous infusion, transdermal patches, etc.

The term “ameliorating” or “ameliorate” refers to any indicia of success in the treatment of a pathology or condition, including any objective or subjective parameter such as abatement, remission or diminishing of symptoms or an improvement in a patient's physical or mental well-being. Amelioration of symptoms can be based on objective or subjective parameters, including the results of a physical examination and/or a psychiatric evaluation.

The term “therapy” refers to “treating” or “treatment” of a disease or condition including preventing the disease or condition from occurring in an animal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease).

The term “effective amount” or “an amount effective to” or a “therapeutically effective amount” or any grammatically equivalent term means the amount that, when administered to an animal for treating a disease, is sufficient to effect treatment for that disease.

The term “enhanced” refers to any degree of betterment, augmentation embellishment, beautification, strengthening and/or improvement. For example, the phrase “enhanced performance” indicates that performance is improved in one state, by comparison to another.

The term “improved” refers to a more desirable condition than previously existed, or alternatively, improved refers to state wherein a more desirable result is achieved under one set of conditions as compared with another. Improvement is demonstrated by any indicia of success, betterment, progression, or amelioration including any objective or subjective parameter such as abatement, remission, and/or diminishing of symptoms or an improvement in an individual's physical or mental well-being. Improvement can be based on objective or subjective parameters, including the results of a physical examination and/or a psychiatric evaluation.

The phrase “functional effects” in the context of assays for testing compounds affecting a osteopontin activity includes the determination of any parameter that is indirectly or directly under the influence of the osteopontin. It includes changes calcification or anti-calcification activity, and also includes other physiologic effects such as increases or decreases cell migration or inflammatory processes.

Introduction

In one aspect, the present invention provides a constitutively phosphorylated osteopontin peptide. Osteopontin is the principal phosphorylated glycoprotein of bone, but is also expressed in other tissues including dentine. In bone during bone development, osteopontin is produced by matrix-producing osteoblasts at the mineralization front. Under stimulation by calcitriol (1,25-dihydroxy-vitamin(D3)), osteopontin is secreted by bone resorbing osteoclasts.

Osteopontin facilitates the attachment of osteoblasts and osteoclasts to the extracellular matrix, allowing them to perform their respective functions during osteogenesis (Reinholt et al. (1990) Proc. Natl. Acad. Sci. U.S.A. 87:4473-5). Several other functions have been suggested for this protein, including the role of an inflammatory cytokine in rheumatoid synovitis (Xu G. et al., (2005) J. Clin. Invest. 115:1060-1067), and as a regulator of cell migration.

Osteopontin is up-regulated in association with various disease states related to calcification, including arterial plaque formation, the formation of kidney stones (Beck G. R. Jr, et al. (2000) Proc. Natl. Acad. Sci. U.S.A. 97:8352-7) and in response to tissue injury and/or the implantation of prosthetic devices.

The osteopontin gene comprises 7 exons, 6 of which contain coding sequence (Crosby et al., (1995) Genomics 27:155-60). The protein comprises some highly conserved sequences such as a cell adhesion domain, DGRGDSVAYG (SEQ ID NO:2) and a heparin binding homology domain RKKRSKKFRR (SEQ ID NO:3).

Phosphorylation is an important factor which influences the activity of the peptide in calcification processes. Indeed, depending on the phosphorylation state of the peptide, osteopontin can either inhibit, or promote calcification. For example, Jono et al. (2000, JBC 275:20197) have shown that phosphorylated osteopontin inhibits calcification, and further, that the degree of inhibition is proportional to the number of phosphorylated sites on the protein.

Synthesis of the Peptides and Methods of the Invention

One of skill in the art given the following disclosure and the knowledge in the art will find that techniques useful in synthesizing the constitutively phosphorylated osteopontin peptides of the invention are both readily apparent and accessible.

Isolating the Gene Encoding Osteopontin

A. General Recombinant DNA Methods

This invention relies on routine techniques in the field of recombinant genetics. Basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized according to the solid phase phosphoramidite triester method first described by Beaucage & Caruthers, (1981) Tetrahedron Letts. 22:1859-1862, using an automated synthesizer, as described in Van Devanter et. al., (1984) Nucleic Acids Res. 12:6159-6168. Purification of oligonucleotides is by either native acrylamide gel electrophoresis or by anion-exchange HPLC as described in Pearson & Reanier, (1983) J. Chrom. 255:137-149.

The sequence of the cloned genes and synthetic oligonucleotides can be verified after cloning using, e.g., the chain termination method for sequencing double-stranded templates of Wallace et al., Gene 16:21-26 (1981).

B. Cloning Methods for the Isolation of Nucleotide Sequences Encoding Osteopontin

In general, the nucleic acid sequences encoding osteopontin and related nucleic acid sequence homologs are cloned from cDNA and genomic DNA libraries or isolated using amplification techniques with oligonucleotide primers. For example, osteopontin sequences are typically isolated from human nucleic acid (genomic or cDNA) libraries by hybridizing with a nucleic acid probe or polynucleotide, the sequence of which can be derived from SEQ ID NO:1. Suitable tissues from which osteopontin RNA and cDNA can be isolated include, but are not limited to, osteoblast and osteoclast cells and cell lines, kidney tissue, atherosclerotic tissue, human and bovine milk, and various tumors.

Amplification techniques using primers can also be used to amplify and isolate osteopontin from DNA or RNA. The following exemplary primers can be used to amplify a sequence of osteopontin:

Forward primer:
5′ AACGCCGACC AAGGAAAACT CACTACC 3′
Reverse primer:
5′ CTCCTTTTAA TTGACCTCAG AAGATGC 3′

The human osteopontin cDNA sequence used for design the primers is from Kiefer M C et al (1989) Nucleic Acids Research 17(8): 3306. These primers can be used, e.g., to amplify either the full length sequence or a probe of one to several hundred nucleotides, which is then used to screen a human library for full-length osteopontin.

Nucleic acids encoding osteopontin can also be isolated from expression libraries using antibodies as probes. Such polyclonal or monoclonal antibodies can be raised using the sequence of SEQ ID NO:1.

Human osteopontin polymorphic variants, orthologs, and alleles that are substantially identical to osteopontin can be isolated using osteopontin nucleic acid probes and oligonucleotides under stringent hybridization conditions, by screening libraries e.g., cDNA libraries. Alternatively, expression libraries may be used to clone osteopontin and osteopontin polymorphic variants, orthologs, and alleles by detecting expressed homologs immunologically with antisera or purified antibodies made against osteopontin or portions thereof which also recognize and selectively bind to the osteopontin homolog. To bypass the potential difficulty in separating polymorphic variants with single amino acid differences, a more practical or feasible approach to produce modified osteopontin would involve the use of a cell line that does not natively express osteopontin.

To make a cDNA library, one should choose a source that is rich in osteopontin mRNA, e.g., tissue including but not limited to, osteoblast and osteoclast cells and cell lines, kidney tissue, atherosclerotic, and various tumors. As is known in the art, the mRNA is then made into cDNA using reverse transcriptase, ligated into a recombinant vector, and transfected into a recombinant host for propagation, screening and cloning. Indeed, methods for making and screening cDNA libraries are well known (see, e.g. Gubler & Hoffman (1983) Gene 25:263-269; Sambrook et al., supra; Ausubel et al., supra).

For a genomic library, the DNA is extracted from the tissue and either mechanically sheared or enzymatically digested to yield fragments of about 12-20 kb. The fragments are then separated by gradient centrifugation from undesired sizes and are constructed in bacteriophage lambda vectors. These vectors and phage are packaged in vitro. Recombinant phage is analyzed by plaque hybridization as described in Benton & Davis, (1977) Science 196:180-182. Colony hybridization is carried out as generally described in Grunstein et al., (1975) Proc. Natl. Acad. Sci. U.S.A., 72:3961-3965.

An alternative method of isolating osteopontin nucleic acid and its homologs combines the use of synthetic oligonucleotide primers and amplification of an RNA or DNA template (see U.S. Pat. Nos. 4,683,195 and 4,683,202; PCR Protocols: A Guide to Methods and Applications (Innis et al., eds, 1990)). Methods such as polymerase chain reaction (PCR) and ligase chain reaction (LCR) can be used to amplify nucleic acid sequences of osteopontin directly from mRNA, from cDNA, from genomic libraries or cDNA libraries. Degenerate oligonucleotides can be designed to amplify osteopontin homologs using the sequences provided herein. Restriction endonuclease sites can be incorporated into the primers. Polymerase chain reaction or other in vitro amplification methods may also be useful, for example, to clone nucleic acid sequences that code for proteins to be expressed, to make nucleic acids to use as probes for detecting the presence of osteopontin encoding mRNA in physiological samples, for nucleic acid sequencing, or for other purposes. Genes amplified by the PCR reaction can be purified from agarose gels and cloned into an appropriate vector.

Gene expression of osteopontin can also be analyzed by techniques known in the art, e.g., reverse transcription and amplification of mRNA, isolation of total RNA or poly A+ RNA, northern blotting, dot blotting, in situ hybridization, RNase protection, high density polynucleotide array technology and the like.

Synthetic oligonucleotides can be used to construct recombinant osteopontin genes for use as probes or for expression of protein. This method is performed using a series of overlapping oligonucleotides usually 40-120 bp in length, representing both the sense and antisense strands of the gene. These DNA fragments are then annealed, ligated and cloned. Alternatively, amplification techniques can be used with precise primers to amplify a specific subsequence of the osteopontin gene. The specific subsequence is then ligated into an expression vector.

The gene for osteopontin is typically cloned into intermediate vectors before transformation into prokaryotic or eukaryotic cells for replication and/or expression. These intermediate vectors are typically prokaryote vectors, e.g., plasmids, or shuttle vectors.

C. Preparing Mutant Forms of Osteopontin

Once the osteopontin peptide sequences are obtained, mutant forms of osteopontin can be generated using techniques well known in the art (see e.g., Sambrook et al., supra; Ausubel et al., supra).

In an exemplary embodiment, a mutation that changes a serine residue or a threonine residue to a cysteine residue is useful for creating a constitutively phosphorylated osteopontin. In some embodiments, the serine or threonine residue that is mutated to cysteine is a phosphorylatable serine or threonine residue. Another approach would be to replace one or more serine residues, e.g. those integral to reduced calcification, with aspartic acid (see Huang W and Erickson R L. (1994) Proc. Natl. Acad. Sci. USA 91: 8960-8963).

In another exemplary embodiment, mutations are introduced into an osteopontin gene sequence, such that the resulting expressed peptide comprises new phosphorylation sites not present in the wild type osteopontin peptide. To explain, phosphorylation of the protein can introduce a conformational change, i.e. a change in its 3-D structure, such that it can bind to different receptors or downstream target protein or other molecules. This can then initiate a chain of molecular response by signal transduction. Generally, another effect of protein phosphorylation is to change its activity after phosphorylation. Examples of such proteins are kinases or proteins with enzymatic activities. As such, phosophorylation of osteopontin can bestow new characteristics on the protein by changing either its conformation or activity.

D. Expression in Prokaryotes and Eukaryotes

To obtain high level expression of a cloned gene, e.g., a high level of expression of a cDNA encoding osteopontin, one typically subclones the osteopontin cDNA into an expression vector that contains a strong promoter to direct transcription, a transcription/translation terminator, and if for a nucleic acid encoding a protein, a ribosome binding site for translational initiation. Suitable bacterial promoters are well known in the art and described, e.g., in Sambrook et al. and Ausubel et al, supra. Bacterial expression systems for expressing the osteopontin protein are available e.g., E. coli, Bacillus sp., and Salmonella (Palva et al., (1983) Gene 22:229-235; Mosbach et al., (1983) Nature 302:543-545). Kits for such expression systems are commercially available. Eukaryotic expression systems for mammalian cells, yeast, and insect cells are well known in the art and are also commercially available.

Selection of the promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the osteopontin encoding nucleic acid in host cells. A typical expression cassette thus contains a promoter operably linked to the nucleic acid sequence encoding osteopontin and signals required for efficient polyadenylation of the transcript, ribosome binding sites, and translation termination. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺pMTO10/A⁺, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase and dihydrofolate reductase. Alternatively, high yield expression systems not involving gene amplification are also suitable, such as using a baculovirus vector in insect cells, with an osteopontin encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical; any of the many resistance genes known in the art are suitable. The prokaryotic sequences are preferably chosen such that they do not interfere with the replication of the DNA in eukaryotic cells, if necessary.

Standard transformation methods are used to produce bacterial, mammalian, yeast or insect cell lines that express large quantities of osteopontin protein, which are then purified using standard techniques (see, e.g., Colley et al., (1989) J. Biol. Chem. 264:17619-17622; Guide to Protein Purification, in Methods in Enzymology, vol. 182 (Deutscher, ed., 1990)). Transformation of eukaryotic and prokaryotic cells are performed according to standard techniques (see, e.g., Morrison, (1977) J. Bact. 132:349-351; Clark-Curtiss & Curtiss, Methods in Enzymology 101:347-362 (Wu et al., eds, 1983).

Any of the well-known procedures for introducing foreign nucleotide sequences into host cells may be used. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing osteopontin.

After the expression vector is introduced into the cells, the transfected cells are cultured under conditions favoring expression of osteopontin, which is recovered from the culture using standard techniques identified below.

II. Purification of Osteopontin Polypeptides

Either naturally occurring or recombinant osteopontin can be purified for use in functional assays. Naturally occurring osteopontin peptides can be purified, e.g., from human tissue including, but are not limited to, osteoblast and osteoclast cells and cell lines, kidney tissue, atherosclerotic tissue, various tumors, and any other source of an osteopontin peptide. Recombinant osteopontin peptides can be purified from any suitable expression system.

The osteopontin peptides may be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., supra).

Alternatively, osteopontin peptides can be purified by virtue of the integrin binding domain present in the full length peptide. The osteopontin peptides can be selectively adsorbed to a purification column comprising a ligand to which the integrin domain binds. The bound peptides are then freed from the column in a relatively pure form. Finally the osteopontin peptides can be purified using immunoaffinity columns.

B. Purification of Osteopontin Peptides from Recombinant Bacteria

Recombinant proteins expressed by transformed bacteria may be produced in large amounts, typically after promoter induction e.g., IPTG induction of a Lac promoter; but expression can be constitutive. Bacteria are grown according to standard procedures very well known in the art, and the peptides are isolated either from fresh or frozen bacteria cultures.

Unfortunately, proteins expressed in bacteria may form insoluble aggregates (“inclusion bodies”). Several protocols are suitable for purification of the osteopontin peptides from inclusion bodies. For example, purification of inclusion bodies typically involves the extraction, separation and/or purification of inclusion bodies by disruption of bacterial cells, e.g., by incubation in a buffer of 50 mM TRIS/HCL pH 7.5, 50 mM NaCl, 5 mM MgCl₂, 1 mM DTT, 0.1 mM ATP, and 1 mM PMSF. The cell suspension can be lysed using 2-3 passages through a French Press, homogenized using a Polytron (Brinkman Instruments) or sonicated on ice. Alternate methods of lysing bacteria are apparent to those of skill in the art (see, e.g., Sambrook et al., supra; Ausubel et al., supra).

If necessary, the inclusion bodies are solubilized, and the lysed cell suspension is typically centrifuged to remove unwanted insoluble matter. Proteins that formed the inclusion bodies may be renaturized by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents which are capable of solubilizing aggregate-forming proteins, for example SDS (sodium dodecyl sulfate), 70% formic acid, are inappropriate for use in this procedure due to the possibility of irreversible denaturization of the proteins, accompanied by a lack of immunogenicity and/or activity. Although guanidine hydrochloride and similar agents are denaturants, this denaturization is reversible and renaturization may occur upon removal (by dialysis, for example) or dilution of the denaturant, allowing re-formation of immunologically and/or biologically active protein. Other suitable buffers are known to those skilled in the art. Human osteopontin peptides are separated from other bacterial proteins by standard separation techniques, e.g., using size differential filtration and/or column chromatography.

In some embodiments, the osteopontin peptide is isolated from the bacterial periplasm. In this embodiment, the periplasmic fraction of the bacterial culture is isolated by cold osmotic shock as is well known to those of skill in the art. The periplasmic proteins are separated from the cellular debris and from other, unwanted proteins, by centrifugation such that the recombinant proteins of the periplasmic fraction are present in supernatant. The osteopontin peptides remaining in the supernatant are then separated from the remaining contaminating host proteins by standard separation techniques e.g., size differential filtration and/or column chromatography as well as with other techniques well known to those of skill in the art.

B. Size Differential Filtration

The molecular weight of an osteopontin peptide can be used to isolate it from proteins of greater and lesser size using ultrafiltration through membranes of different pore size (for example, Amicon or Millipore membranes). As a first step, the protein mixture is ultra-filtrated through a membrane with a pore size that has a lower molecular weight cut-off than the molecular weight of the protein of interest. The retentate of the ultrafiltration is then ultrafiltrated against a membrane with a molecular cut off greater than the molecular weight of the protein of interest. The recombinant protein will pass through the membrane into the filtrate. The filtrate can then be chromatographed as described below.

C. Column Chromatography

The osteopontin peptides can also be separated from other proteins on the basis of its size, net surface charge, hydrophobicity, and affinity for ligands. In addition, antibodies raised against proteins (see below) can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

III. Immunological Detection of Osteopontin

In addition to the detection of osteopontin genes and gene expression using nucleic acid hybridization technology, one can also use immunoassays to detect the osteopontin peptides. Immunoassays can be used to qualitatively or quantitatively analyze the osteopontin peptides. A general overview of the applicable technology can be found in Harlow & Lane, Antibodies: A Laboratory Manual (1988).

A. Antibodies to Osteopontin Peptides

Methods of producing polyclonal and monoclonal antibodies that react specifically with peptides e.g., osteopontin peptides are known to those of skill in the art (see, e.g., Coligan, Current Protocols in Immunology (1991); Harlow & Lane, supra; Goding, Monoclonal Antibodies: Principles and Practice (2d ed. 1986); and Kohler & Milstein, (1975) Nature 256:495-497. Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., (1989) Science 246:1275-1281; Ward et al, (1989) Nature 341:544-546).

B. Immunological Binding Assays

The osteopontin peptides can be detected and/or quantified using any of a number of well recognized immunological binding assays (see, e.g., U.S. Pat. Nos. 4,366,241; 4,376,110; 4,517,288; and 4,837,168). For a review of the general immunoassays, see also Methods in Cell Biology: Antibodies in Cell Biology, volume 37 (Asai, ed. 1993); Basic and Clinical Immunology (Stites & Terr, eds., 7th ed. 1991). Immunological binding assays (or immunoassays) typically use an antibody that specifically binds to a protein or antigen of choice (in this case the osteopontin or an antigenic subsequence thereof). The antibody (e.g., anti-osteopontin) may be produced by any of a number of means well known to those of skill in the art and as described above.

IV Methods of Making Constitutively Phosphorylated Osteopontin

A. Phosphorylation of Osteopontin

Both natural and recombinant osteopontin can be modified by phosphorylation of the amino acid sequence encoding native osteopontin. The osteopontin can be modified so that phosphorylation is present in the absence of or with altered glycosylation. The osteopontin can also be modified so that it has less phosphorylation or more phosphorylation than wild type forms of osteopontin, or is phosphorylated at sites other than those which are naturally phosphorylated.

In an exemplary embodiment, one or more point mutations and/or and deletions disrupts a regulatory interaction that controls basal phosphorylation state of the osteopontin molecule, resulting in the constitutive phosphorylation in cells. For example, the recognition site for a kinase may be altered to increase activity of the kinase at that site. Thus, the mutation(s) alters the recognition site such that the site is recognized more efficiently than the wild type site.

In another exemplary embodiment, the mutation(s) alters an interaction between an osteopontin peptide a regulatory protein that typically inhibits the action of the kinase see e.g., Barroga, C. F., et al (1995) PNAS USA 92:7637. Disruption of the regulatory interaction by mutation results the constitutive phosphorylation of the osteopontin peptide. In still other exemplary embodiments, the mutation(s) alters the amino acid sequence of the osteopontin peptide to create and/or delete recognition sites relative to the sequence of the wild type osteopontin peptide.

In still another exemplary embodiment, the concentration of a kinase is effectively increased, thereby effectively increasing the activity of the kinase. For example, effectively increasing the concentration of any given kinase in a cell is achieved e.g., by overexpressing the kinase in the cell.

1. Protein Kinase Recognition Sites

The determinants of protein kinase specificity involve complex 3-dimensional interactions. In exemplary embodiments, osteopontin peptides are phosphorylated by naturally occurring kinases and/or by recombinant kinases to produce constitutively phosphorylated osteopontin peptides. In other exemplary embodiment, an osteopontin peptide is mutated

Short amino-acid sequence motifs which describe the primary structure around the phosphoacceptor residue for a particular protein kinase describe the recognition elements for the kinase. Understanding of the recognition sites for various protein kinases can be used to selectively design constitutively phosphorylated osteopontin peptides. The sequence motifs for the various kinase recognition sites are useful in creating or identifying protein kinase recognition sites and, hence, phosporylation sites.

Table 1 summarizes the recognition sequences of some exemplary well-studied protein kinases (for a further list see e.g., Pearson, R. B., and Kemp, B. E. (1991). In T. Hunter and B. M. Sefton (Eds.), Methods in Enzymology Vol. 200, (pp. 62-81). San Diego: Academic Press, and New England BioLabs catalog each of which are incorporated herein by reference in their entirety). Phosphoacceptor residue is indicated in italics, amino acids which can function interchangeably at a particular residue are separated by a slash (/), and residues which do not appear to contribute strongly to recognition are indicated by an “X”. This list of kinases is meant to be exemplary and is by no means intended to limit the invention. Indeed, any protein kinase whose recognition site has been identified is suitable for use in the invention.

TABLE 1
PROTEIN KINASE RECOGNITION SEQUENCES

| | Recognition | Phosphorylation | Protein Substrate |
|---|---|---|---|
| Protein Kinase | Motifs | Sites | (reference) |
| cAMP-dependent Protein Kinase | R-X-S/T | Y₇LRRASLAQLT | pyruvate kinase (2) |
| (PKA, cAPK) | R-R/K-X-S/T | F₁RRLSIST | phosphorylase kinase, |
| | | A₂₉GARRKASGPP | α chain (2) |
| | | | histone H1, bovine (2) |
| Casein Kinase I (CKI, CK-1) | S(P)-X-X-S/T | R₄TLS(P)VSSLPGL | glycogen synthase, |
| | | D₄₃IGS(P)ES(P)TEDQ | rabbit muscle (4) |
| | | | αₛ₁-casein (4) |
| Casein Kinase II (CKII, CK-2) | S/T-X-X-E | A₇₂DSESEDEED | PKA regulatory subunit, |
| | | L₃₇ESEEEGVPST | R_{II} (2) |
| | | E₂₆DNSEDEISNL | p34^{cdc2}, human (5) |
| | | | acetyl-CoA carboxylase |
| | | | (2) |
| Glycogen Synthase Kinase 3 (GSK-3) | S-X-X-X-S(P) | S₆₄₁VPPSPSLS(P) | glycogen synthase, |
| | | S₆₄₁VPPS(P)PSLS(P) | human (site 3b) (6, 2) |
| | | | glycogen synthase, |
| | | | human (site 3a) (6, 2) |
| Cdc2 Protein Kinase; CDK2-cyclin A | S/T-P-X-R/K | P₁₃AKTPVK | histone H1, calf thymus |
| | | H₁₂₂STPPKKKRK | (2) |
| | | | large T antigen (2) |
| Calmodulin-dependent Protein Kinase | R-X-X-S/T | N₂YLRRRLSDSN | synapsin (site 1) (2) |
| II (CaMK II) | R-X-X-S/T-V | K₁₉₁MARVFSVLR | calcineurin (2) |
| Mitogen-activated Protein Kinase | P-X-S/T-P | P₂₄₄LSP | c-Jun (7) |
| (Extracellular Signal-regulated | X-X-S/T-P | P₉₂SSP | cyclin B (7) |
| Kinase) (MAPK, Erk) | | V₄₂₀LSP | Elk-1 (7) |
| Abl Tyrosine Kinase | I/V/L-Y-X-X-P/F | | |

Some protein kinases known in the art are referred to as “hierarchical” protein kinases. These kinases require prior phosphorylation by another kinase at a residue in the vicinity of their own phosphorylation site prior to being activated themselves. In Table 1, these protein kinases are indicated as S(P) to identify the preexisting phosphoserine residues.

Phosphorylation of osteopontin is achieved by incubation of the osteopontin in the presence of any kinases that will result in an osteopontin peptide that is phosphorylated such that the resulting peptide has the activity of a constitutively phosphorylated osteopontin. For example, phosphorylation of human osteopontin (SEQ ID NO:1) using casein kinase II, will result in a peptide phosphorylated at a number of sites, including, but not necessarily limited to, serine 26, 27, 62, 63, 76, 78, 105, 108, 120, 126, 129, 191, 234, 280, 291, 308, and threonine 185.

Kinases can be obtained from cytosolic or microsomal extracts, or in purified or semi-purified form from sources such as Sigma Chemical Co., Inc., or as described in the literature. As described in the example below, at least three different kinase preparations from mouse kidney could be used to phosphorylate osteopontin in vitro. These preparations contain a mixture of kinase activities, several of which can phosphorylate the osteopontin protein. Casein kinase I, casein kinase II and mammary gland casein kinase participate in hierarchical phosphorylation reactions. As noted above, phosphorylation of one hierarchical site by any of these kinases may affect phosphorylation at another site by a different kinase.

Osteopontin appears to be a complex substrate with at least 58 consensus phosphorylation sites for different types of kinases, as shown in Table 2.

TABLE 2
PREDICTED PHOSPHORYLATION SITES IN OSTEOPONTIN

| | 1. Position of |
|---|---|
| 2. Protein | phosphorylated |
| 3. Kinase | residue |
| 4. Casein Kinase I | 239, 275, 280, 308 |
| | 1. 26, 76, 78, 99, 102, |
| | 2. 105, 108, 117, 120, |
| | 3. 123, 126, 129, 234, |
| | 4. 308 |
| 5. Casein Kinase II | 26, 27, 62, 63, 191, |
| | 1. 215, 228, 280, 291 |
| | 2. 76, 237 |
| 6. Ca/Calmodulin- | 162, 171 |
| 7. dependent | |
| 8. Protein Kinase II | |
| 9. cGMP-Dependent | 24, 73, 81, 162, |
| 10. Protein Kinase | 169, 171, 243, 270, |
| | 1. 275, 303 |
| 11. cAMP-Dependent | 224, 243, 270 |
| 12. Protein Kinase | |
| 13. Protein Kinase C | 49, 239, 171 |
| 14. Tyrosine Kinase | 165 |
| 15. Proline-Dependent | 147 |
| 16. Protein Kinase | |

Phosphorylation sites are not randomly distributed throughout the protein but instead are organized in eight clusters. For example, between residues 100 and 126 there are 9 potential phosphorylation sites for either casein kinase I, casein kinase II or mammary gland casein kinase. In addition to containing potential phosphorylation sites for these independent casein kinase family of enzymes, osteopontin also contains potential phosphorylation sites for cAMP- and cGMP-dependent protein kinases, calmodulin-dependent protein kinase, and protein kinase C.

There are several fold more potential phosphorylation sites in recombinant osteopontin than those found phosphorylated in osteopontin isolated from bone. Not all of the potential sites may be phosphorylated at a given time, since some sites may be not accessible to protein kinases or some tissues may not contain all of the kinase activities required for the phosphorylation of osteopontin.

Certain phosphorylated residues can serve as specificity determinants. For example, phosphorylation of a Ser/Thr residue by any kinase can generate a site for phosphorylation of an adjacent phosphorylatable residue by either casein kinase I or mammary gland casein kinase. Conversely, phosphorylation at one site by a particular kinase may suppress the phosphorylation of a nearby residue, such as the mutually exclusive phosphorylation of hormone-sensitive lipase by cAMP-dependent protein kinase and calmodulin-dependent protein kinase. Thus, deletion of some phosphorylation sites which are typically present in the wild type peptide results in increased phosphorylation at nearby phosphorylation sites. Serine 24, 81, 224, and 270 are some exemplary candidate sites for such a deletion approach.

In general, a constitutively phosphorylated osteopontin is phosphorylated at the following sites: serine 26, 27, 62, 63, 76, 78, 105, 108, 120, 126, 129, 191, 234, 280, 291, 308, and threonine 185. Other variations include, but are not limited to serine 24, 81, 224, and 270.

In one exemplary embodiment, phosphorylation is carried out using calcium/calmodulin kinase II, in the presence of 1.5 mM CaCl₂ and 3 μg calmodulin. In another exemplary embodiment, osteopontin is phosphorylated by protein kinase C in the presence of μg/ml phosphatidylserine, 0.8 μg/ml of diacylglycerol, and 1 mM CaCl₂. In another exemplary embodiment, autophosphorylation of osteopontin is carried out in the presence of 10 mM MnCl₂. In another exemplary embodiment, osteopontin is phosphorylated by cGMP dependent protein kinase in the presence of 0.1 μM cGMP.

In another exemplary embodiment, the degree of expression of the a kinase nucleic acid in the cell line is high enough to result in constitutive phosphorylation of the osteopontin.

In an exemplary embodiment, phosphorylation sites are created or modified e.g., by creating a new phosphorylation site, or e.g., deleting an existing phosphorylation site, and/or e.g., by creating sites comprising phophorylatable cysteine residues such that the resulting mutated peptide is constitutively phosphorylated.

In another exemplary embodiment, consensus phosphorylation sequences are identified and phosphorylated with kinases known to phosphorylate the concensus sequence. For example, osteopontin sequences can be selected to comprise substantially the sequence of the casein kinase II phosphorylation consensus region, SGSSEEK (SEQ ID NO:4), or the C-terminal heparin binding homology domain SKEEDKHLKFRISHELDSASSEVN (SEQ ID NO:5) which contains three conserved sites of serine phosphorylation. In some embodiments, an osteopontin peptide may alternatively or additionally include the alternatively spliced domain, NAVSSEETNDFKQE (SEQ ID NO:6), which contains two sites of serine phosphorylation. Additional sites of serine and threonine phosphorylation are described, for example, in Sorensen et al., (1994) Biochem. Biophys. Res. Comm. 198:200-205.

2. Constitutive Phosphorylation of Osteopontin Using ATPγS

In certain exemplary embodiments, an osteopontin peptide is irreversibly phosphorylated by incubating the cells expressing an osteopontin peptide in the presence of the ATP analog, ATP gamma S or [³⁵S]ATPγS. Cells incubated in the presence of [³⁵S]ATPγS produce thiophosphorylated osteopontin peptides that are resistant to dephosphorylation with phosphatase. Thus, once a phosphate is added, it is not removed. Alternatively, purified osteopontin peptides of the invention can be thiophosphorylated in vitro by methods known in the art.

V. Determination of Phosphorylated Sites in Osteopontin

A. Proteolytic Digestion and Chromatography

In an exemplary embodiment, after phosphorylation with ³²P-ATP or [³⁵S]ATPγS and the desired kinase, osteopontin is digested with either trypsin, endopeptidase Glu-C, or endopeptidase Asp-N. The resulting peptides are separated by HPLC and the radiolabeled peptides sequenced. The position of the phosphorylated residue is determined by the co-elution of radioactivity with the amino acid in that cycle.

B. Dephosphorylation of Osteopontin

In another exemplary embodiment, osteopontin can be dephosphorylated by incubating the protein in either 100 μl 20 mM HEPES buffer, pH 8.5, and 1 unit of alkaline phosphatase, or 100 μl 20 mM acetate buffer pH, 5.0 and 1 unit of acid phosphatase, for several hours. Osteopontin can also be dephosphorylated by incubating the phosphoprotein with between 0.1 and 1 units of protein phosphatase 2A at 4° C. for 1 h. Osteopontin can be also dephosphorylated by incubating the protein in 0.1 N NaOH for 1 h at 37° C.

VI. Cellular Transfection and Gene Therapy

In exemplary embodiments, the present invention provides nucleic acids encoding osteopontin that are used for the transfection of cells in vitro and in vivo. These nucleic acids can be inserted into any of a number of well-known vectors for the transfection of target cells and organisms as described below. The nucleic acids are transfected into cells, ex vivo or in vivo, through the interaction of the vector and the target cell. The nucleic acid for osteopontin, under the control of a promoter, then expresses an osteopontin monomer of the present invention, thereby mitigating the effects of absent, partial inactivation, or abnormal expression of the osteopontin gene.

Such gene therapy procedures have been used to correct acquired and inherited genetic defects, cancer, and viral infection in a number of contexts. The ability to express artificial genes in humans facilitates the prevention and/or cure of many important human diseases, including many diseases which are not amenable to treatment by other therapies (for a review of gene therapy procedures, see Anderson, (1992) Science 256:808-813; Nabel & Felgner, (1993) TIBTECH 11:211-217; Mitani & Caskey, (1993) TIBTECH 11:162-166; Mulligan, (1993) Science 260(5110):926-932; Dillon, (1993) TIBTECH 11:167-175; Miller, (1992) Nature 357:455-460; Van Brunt, (1998) Biotechnology 6(10):1149-1154; Vigne, (1995) Restorative Neurology and Neuroscience 8:35-36; Kremer & Perricaudet, (1995) British Medical Bulletin 51(1):31-44; Haddada et al., in Current Topics in Microbiology and Immunology (Doerfler & Bohm eds., 1995); and Yu et al., (1994) Gene Therapy 1:13-26).

Delivery of the gene or genetic material into the cell is the first critical step in gene therapy treatment of disease. A large number of delivery methods are well known to those of skill in the art. Preferably, the nucleic acids are administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell.

Methods of non-viral delivery of nucleic acids include lipofection, microinjection, biolistics, virosomes, liposomes, immunoliposomes, polycation or lipid:nucleic acid conjugates, naked DNA, artificial virions, and agent-enhanced uptake of DNA. Lipofection is described in, e.g., U.S. Pat. Nos. 5,049,386, 4,946,787; and U.S. Pat. No. 4,897,355 and lipofection reagents are sold commercially (e.g., Transfectam™ and Lipofectin™). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, (1995) Science 270:404-410; Blaese et al., (1995) Cancer Gene Ther. 2:291-297; Behr et al., (1994) Bioconjugate Chem. 5:382-389; Remy et al., (1994) Bioconjugate Chem. 5:647-654; Gao et al., (1995) Gene Therapy 2:710-722; Ahmad et al., (1992) Cancer Res. 52:4817-4820; U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vector that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), simian immunodeficiency virus (SIV), human immunodeficiency virus (HIV), and combinations thereof (see, e.g., Buchscher et al., (1992) J. Virol. 66:2731-2739; Johann et al., (1992) J. Virol. 66:1635-1640; Sommerfelt et al., (1990) Virol. 176:58-59; Wilson et al., (1989) J. Virol. 63:2374-2378; Miller et al., (1991) J. Virol. 65:2220-2224; PCT/US94/05700).

In applications where transient expression of the nucleic acid is preferred, adenoviral based systems are typically used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., (1987) Virology 160:38-47; U.S. Pat. No. 4,797,368; WO 93/24641; Kotin, (1994) Human Gene Therapy 5:793-801; Muzyczka, (1994) J. Clin. Invest. 94:1351). Construction of recombinant AAV vectors are described in a number of publications, including U.S. Pat. No. 5,173,414; Tratschin et al., (1985) Mol. Cell. Biol. 5:3251-3260; Tratschin et al., (1984) Mol. Cell. Biol. 4:2072-2081; Hermonat & Muzyczka, (1984) Proc. Natl. Acad. Sci. U.S.A. 81:6466-6470; and Samulski et al., (1989) J. Virol. 63:03822-3828.

In particular, at least six viral vector approaches are currently available for gene transfer in clinical trials, with retroviral vectors being by far the most frequently used system. All of these viral vectors utilize approaches that involve complementation of defective vectors by genes inserted into helper cell lines to generate the transducing agent.

pLASN and MFG-S are examples are retroviral vectors that have been used in clinical trials (Dunbar et al., (1995) Blood 85:3048-305; Kohn et al., (1995) Nat. Med. 1:1017-102; Malech et al., (1997) Proc. Natl. Acad. Sci. U.S.A. 94:22 12133-12138). PA317/pLASN was the first therapeutic vector used in a gene therapy trial. (Blaese et al., (1995) Science 270:475-480). Transduction efficiencies of 50% or greater have been observed for MFG-S packaged vectors (Ellem et al., (1997) Immunol Immunother. 44(1): 10-20; Dranoff et al., (1997) Hum. Gene Ther. 1:111-2).

Recombinant adeno-associated virus vectors (rAAV) are a promising alternative gene delivery system based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system (Wagner et al., (1998) Lancet 351:9117 1702-3, Kearns et al., (1996) Gene Ther. 9:748-55).

Replication-deficient recombinant adenoviral vectors (Ad) are predominantly used transient expression gene therapy, because they can be produced at high titer and they readily infect a number of different cell types. Most adenovirus vectors are engineered such that a transgene replaces the Ad E1a, E1b, and E3 genes; subsequently the replication defector vector is propagated in human 293 cells that supply deleted gene function in trans. Ad vectors can transduce multiple types of tissues in vivo, including nondividing, differentiated cells such as those found in the liver, kidney and muscle system tissues. Conventional Ad vectors have a large carrying capacity. An example of the use of an Ad vector in a clinical trial involved polynucleotide therapy for antitumor immunization with intramuscular injection (Sterman et al., (1998) Hum. Gene Ther. 7:1083-9). Additional examples of the use of adenovirus vectors for gene transfer in clinical trials include Rosenecker et al., (1996) Infection 241:5-10; Sterman et al., (1998) Hum. Gene Ther. 9:7 1083-1089; Welsh et al., (1995) Hum. Gene Ther. 2:205-18; Alvarez et al., (1997) Hum. Gene Ther. 5:597-613; Topf et al., (1998) Gene Ther. 5:507-513; Sterman et al., (1998) Hum. Gene Ther. 7:1083-1089.

Packaging cells are used to form virus particles that are capable of infecting a host cell. Such cells include 293 cells, which package adenovirus, and ψ2 cells or PA317 cells, which package retrovirus. Viral vectors used in gene therapy are usually generated by producer cell line that packages a nucleic acid vector into a viral particle. The vectors typically contain the minimal viral sequences required for packaging and subsequent integration into a host, other viral sequences being replaced by an expression cassette for the protein to be expressed. The missing viral functions are supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the viruses outer surface. The ligand is chosen to have affinity for a receptor known to be present on the cell type of interest. For example, Han et al., (1995) Proc. Natl. Acad. Sci. U.S.A. 92:9747-9751, reported that Moloney murine leukemia virus can be modified to express human heregulin fused to gp70, and the recombinant virus infects certain human breast cancer cells expressing human epidermal growth factor receptor. This principle can be extended to other pairs of virus expressing a ligand fusion protein and target cell expressing a receptor. For example, filamentous phage can be engineered to display antibody fragments (e.g., FAB or Fv) having specific binding affinity for virtually any chosen cellular receptor.

Although the above description applies primarily to viral vectors, the same principles can be applied to nonviral vectors. Such vectors can be engineered to contain specific uptake sequences thought to favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells harvested from an individual patient (e.g., lymphocytes, bone marrow aspirates, tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid (gene or cDNA), and re-infused back into the subject organism (e.g., patient). Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from patients).

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+cells in vitro into clinically important immune cell types using cytokines such a GM-CSF, IFN-.gamma. and TNF-α are known (see Inaba et al., (1992) J. Exp. Med. 176:1693-1702).

Stem cells are isolated for transduction and differentiation using known methods. For example, stem cells are isolated from bone marrow cells by panning the bone marrow cells with antibodies which bind unwanted cells, such as CD4+ and CD8+(T cells), CD45+(panb cells), GR-1 (granulocytes), and lad (differentiated antigen presenting cells) (see Inaba et al., (1992) J. Exp. Med. 176:1693-1702).

Vectors (e.g., retroviruses, adenoviruses, liposomes, etc.) containing therapeutic nucleic acids can be also administered directly to the organism for transduction of cells in vivo. Alternatively, naked DNA can be administered.

Administration is by any of the routes normally used for introducing a molecule into ultimate contact with blood or tissue cells. The nucleic acids are administered in any suitable manner, preferably with pharmaceutically acceptable carriers. Suitable methods of administering such nucleic acids are available and well known to those of skill in the art, and, although more than one route can be used to administer a particular composition, a particular route can often provide a more immediate and more effective reaction than another route.

VIII. Pharmaceutical Compositions

Constitutively phosphorylated osteopontin peptides used in the methods of the invention can be administered by any means known in the art, e.g., parenterally, topically, orally, or by local administration, such as by aerosol or transdermally. The methods of the invention provide for prophylactic and/or therapeutic treatments. The constitutively phosphorylated osteopontin peptides as pharmaceutical formulations can be administered in a variety of unit dosage forms depending upon the condition or disease and the degree and location of the ectopic calcification, the general medical condition of each patient, the resulting preferred method of administration and the like. Details on techniques for formulation and administration are well described in the scientific and patent literature, see, e.g., the latest edition of Remington's Pharmaceutical Sciences, Maack Publishing Co, Easton Pa. (“Remington's”).

Therapeutically effective amounts of constitutively phosphorylated osteopontin peptides suitable for practice of the method of the invention will depend on various factors, including but not limited to, the target tissue site, the nature of constructs, delivery methods, and the manner in which the gene product will be used. A person of ordinary skill in the art will be able, without undue experimentation but having regard to that skill and this disclosure, to determine a therapeutically effective amount of a particular constitutively phosphorylated osteopontin peptide for practice of this invention. For injection into the tissue directly, approximately 25 to 1000 ug per injection at a concentration of 0.2 to 20 mg/ml can be utilized. As for coating a prosthetic device, the precise dosage will depend on the types of device, coating methods and process, etc.

Pharmaceutically acceptable carriers are determined in part by the particular composition being administered (e.g., nucleic acid, protein, modulatory compounds or transduced cell), as well as by the particular method used to administer the composition. Accordingly, there are a wide variety of suitable formulations of pharmaceutical compositions of the present invention (see, e.g., Alfonso A R: Remington's Pharmaceutical Sciences, 17th ed., Mack Publishing Company, Easton Pa., 1989).

For example, compositions may take the form of tablets, pills, capsules, semisolids, powders, sustained release formulations, solutions, suspensions, elixirs, aerosols, or any other appropriate compositions, and comprise at least one compound of this invention in combination with at least one pharmaceutically acceptable excipient. Suitable excipients are well known to persons of ordinary skill in the art, and they, and the methods of formulating the compositions, may be found in such standard references Remington's supra. Suitable liquid carriers, especially for injectable solutions, include e.g. water, aqueous saline solution, aqueous dextrose solution, and glycols.

Aqueous suspensions of the invention contain constitutively phosphorylated osteopontin peptides in admixture with excipients suitable for the manufacture of aqueous suspensions. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Oil suspensions can be formulated by suspending constitutively phosphorylated osteopontin peptides in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, (1997) J. Pharmacol. Exp. Ther. 281:93-102. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

Constitutively phosphorylated osteopontin peptide pharmaceutical formulations can be prepared according to any method known to the art for the manufacture of pharmaceuticals. Such drugs can contain sweetening agents, flavoring agents, coloring agents and preserving agents. Any constitutively phosphorylated osteopontin peptide formulation can be admixed with nontoxic pharmaceutically acceptable excipients which are suitable for manufacture.

Typically, constitutively phosphorylated osteopontin peptides suitable for use in the practice of this invention will be administered by injection. The amount of a compound of this invention in the composition may vary widely depending on the type of composition, size of a unit dosage, kind of excipients, and other factors well known to those of ordinary skill in the art. Another approach is to inject the gene itself, rather the gene product which inevitably degrades once it is placed in the host tissue. For this reason, the injection of the gene product is sufficient for short-term effect whereas injection of the gene can achieve either short-term or long-term effect as the protein, i.e. activated mutant osteopontin, is synthesized by the host cell. In general, the final composition may comprise from 0.000001 percent by weight (% w) to 10% w of the constitutively phosphorylated osteopontin peptides, preferably 0.00001% w to 1% w, with the remainder being the excipient or excipients.

The constitutively phosphorylated osteopontin peptides of the invention can also be delivered as microspheres for slow release in the body. For example, microspheres can be administered via intradermal injection of drug (e.g., constitutively phosphorylated osteopontin peptide-containing microspheres), which slowly release subcutaneously (see Rao, J. Biomater Sci. Polym. Ed. 7:623-645, 1995; as biodegradable and injectable gel formulations (see, e.g., Gao (1995) Pharm. Res. 12:857-863).

The constitutively phosphorylated osteopontin peptide pharmaceutical formulations of the invention can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a pH range of 4.5 to 5.5, that is combined with buffer prior to use.

In another embodiment, the constitutively phosphorylated osteopontin peptide formulations of the invention are useful for intravenous (IV) administration. The formulations for administration will commonly comprise a solution of the constitutively phosphorylated osteopontin peptide dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose, any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of constitutively phosphorylated osteopontin peptide in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the constitutively phosphorylated osteopontin peptide formulations of the invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the constitutively phosphorylated osteopontin peptide into the target cells in vivo. (See, e.g., Al-Muhammed, (1996) J. Microencapsul. 13:293-306; Chonn, (1995) Curr. Opin. Biotechnol. 6:698-708; Ostro, (1989) Am. J. Hosp. Pharm. 46:1576-1587).

After a pharmaceutical comprising a constitutively phosphorylated osteopontin peptide of the invention has been formulated in a acceptable carrier, it can be placed in an appropriate container and labeled for treatment of an indicated condition. For administration of constitutively phosphorylated osteopontin peptides, such labeling would include, e.g., instructions concerning the amount, frequency and method of administration. In one embodiment, the invention provides for a kit for treating ectopic calcification in a subject which includes a constitutively phosphorylated osteopontin peptide composition and instructional material teaching the indications, dosage and schedule of administration of the constitutively phosphorylated osteopontin peptide composition.

IX. Determining Dosing Regimens for Constitutively Phosphorylated Osteopontin

The methods of this invention treat ectopic calcification in a subject. The amount of a constitutively phosphorylated osteopontin peptide adequate to accomplish this is defined as a “therapeutically effective dose”. The dosage schedule and amounts effective for this use, i.e., the “dosing regimen,” will depend upon a variety of factors, including the severity of the ectopic calcification, the patient's physical status, age and the like. In calculating the dosage regimen for a patient, the mode of administration also is taken into consideration.

The dosage regimen also takes into consideration pharmacokinetics parameters, for example the constitutively phosphorylated osteopontin peptide's rate of absorption, bioavailability, metabolism, clearance, and the like (see, e.g., the latest Remington's, supra).

Single or multiple administrations of a constitutively phosphorylated osteopontin peptide formulations can be administered depending on the dosage and frequency as required and tolerated by the patient. The formulations should provide a sufficient quantity of active agent, i.e., a constitutively phosphorylated osteopontin peptide, to effectively treat ectopic calcification in a subject. For example, an exemplary pharmaceutical formulation for injection of a constitutively phosphorylated osteopontin peptide would be about 5 to 15 mg/kg of body weight per patient per day, alternatively between about 8 to about 12 mg/kg of body weight per patient per day, although dosages of between about 0.5 to about 25 mg/kg of body weight per day may be used in the practice of the invention. Lower dosages can also be used. Since ectopic calcification is usually a localized event, it is desirable that the therapy be performed locally to reduce the risk of side effects. Furthermore, greater efficacy can be achieved through delivery of the therapeutic agents (gene or gene product) directly to the diseased spot.

### EXAMPLES

### Example 1

### Creating a Constitutively Phosphorylated Osteopontin

The following example illustrates a method for making a constitutively phosphorylated osteopontin peptide.

Mutations are introduced at sites corresponding to serine 26, 27, 62, 63, 76, 78, 105, 108, 120, 126, 129, 191, 234, 280, 291, 308, and threonine 185 of the osteopontin peptide shown in SEQ ID NO:1. When the mutant peptides are expressed, they function as constitutively phosphorylated osteopontin peptides.

### Example 2

### Treating Ectopic Calcification by Injection of a Constitutively Phosphorylated Osteopontin Peptide at the Site of Implantation

The following example illustrates that the method of the invention is an effective treatment for ectopic calcification.

Constitutively phosphorylated osteopontin from Example 1, is used to treat subjects as described below.

A mouse ectopic calcification model as disclosed in Steitz, S. A. et al (2002) Am J Pathol 161(6):2035-46 is used to demonstrate the efficacy of the invention.

Briefly, all three genotypes of mutant mice homozygous OPN+/+, heterozygous OPN+/− and homozygous OPN−/− are used. As described in Steitz et al., porcine aortic valve leaflets are implanted subcutaneously into the dorsal side of the anesthetized mice.

A subset of mice receive a first injection of constitutively phosphorylated OPN at the time of implantation and this group then receives further daily OPN injections at the implantation site for the duration of the experiment.

At time corresponding to 7 days post implantation, 14 days post implantation and 30 days post implantation and 90 days post implantation, mice are euthanized and examined biochemically and histologically to determine the extent of calcification/mineralization and protein accumulation around the implanted porcine aortic valves.

Successful results are evident as indicated in the table below.

TABLE 3
Calcification of Implanted Porcine Aortic Valves

| | OPN+/+ | OPN+/− | OPN−/− |
|---|---|---|---|
| 7 days | None | None | None |
| No OPN | | | |
| 7 days + | None | None | None |
| OPN | | | |
| 14 days | Low | High | High |
| No OPN | | 4-5X OPN | 4-5X OPN |
| | | +/+ | +/+ |
| 14 days + | None | Low | Low |
| OPN | | | |
| 30 days | Low | High | High |
| No OPN | | 4-5X OPN | 4-5X OPN |
| | | +/+ | +/+ |
| 30 days + | None | Low | Low |
| OPN | | | |
| 90 days | Med | High | High |
| No OPN | | about 3X | about 3X |
| | | OPN +/+ | OPN +/+ |
| 90 days + | Low | Low | Med |
| OPN | | | |

### Example 3

### Preventing Ectopic Calcification Through an Engineered Tissue Valve Expressing a Constitutively Phosphorylated Osteopontin Peptide

The following example illustrates that the method of the invention is an effective treatment for ectopic calcification.

Constitutively phosphorylated osteopontin from Example 1, is used to treat subjects as described below.

A mouse ectopic calcification model as disclosed in Steitz, S. A. et al (2002) 161:2035 is used to demonstrate the efficacy of the invention.

Briefly, all three genotypes of mutant mice homozygous OPN+/+, heterozygous OPN+/− and homozygous OPN−/− are used.

The anti-calcification effect can be achieved using an engineered tissue valve in lieu of coating or pretreating the fixed valve leaflet since the engineered tissue valve includes cells that uptake the osteopontin construct and produce active osteopontin protein. Alternatively, the phosphorylated protein or even cells producing constitutively active osteopontin can be delivered by means of nanotechnology into the valve to prevent or treat a calcification lesion.

At times corresponding to 7 days post implantation, 14 days post implantation and 30 days post implantation and 90 days post implantation, mice are euthanized and examined biochemically and histologically to determine the extent of calcification/mineralization around the implanted porcine aortic valves.

Successful results are evident as indicated in Table 4 below.

TABLE 4
Calcification of OPN Treated Implanted Porcine Aortic Valves

| | OPN+/+ | OPN+/− | OPN−/− |
|---|---|---|---|
| 7 days | None | None | None |
| Gluteraldehyde | | | |
| 7 days | None | None | None |
| Gluteraldehyde + | | | |
| OPN | | | |
| 14 days | Low | High | High |
| Gluteraldehyde | | 4-5X OPN | 4-5X OPN |
| | | +/+ | +/+ |
| 14 days | None | Low | Low |
| Gluteraldehyde + | | | |
| OPN | | | |
| 30 days | Low | High | High |
| Gluteraldehyde | | 4-5X OPN | 4-5X OPN |
| | | +/+ | +/+ |
| 30 days | None | Low | Low |
| Gluteraldehyde + | | | |
| OPN | | | |
| 90 days | Med | High | High |
| Gluteraldehyde | | about 3X | about 3X |
| | | OPN +/+ | OPN +/+ |
| 90 days | Low | Low | Med |
| Gluteraldehyde + | | | |
| OPN | | | |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. An isolated nucleic acid encoding an osteopontin peptide with anti-calcification activity.

2. A peptide encoded by the nucleic acid of claim 1.

3. The peptide of claim 2, wherein the peptide is constitutively phosphorylated.

4. The peptide of claim 2, wherein the constitutively phosphorylated osteopontin comprises at least one thiophosphorylated amino acid.

5. An expression vector comprising the isolated nucleic acid of claim 1 operably linked to a promoter.

6. A cell comprising the isolated nucleic acid of claim 1.

7. The isolated nucleic acid of claim 1, wherein the nucleic acid encodes a mutant osteopontin peptide.

8. A mutant osteopontin peptide according to claim 7.

9. The mutant osteopontin peptide of claim 8, wherein the mutant peptide comprises at least one amino acid substitution with cysteine or aspartic acid for an amino acid which is a member selected from serine 26, 27, 62, 63, 76, 78, 105, 108, 120, 126, 129, 191, 234, 280, 291, 308, and threonine 185.

10. A method of providing therapy for a calcification-related disease to a subject in need thereof, the method comprising:
administering to the subject a therapeutically effective amount of a constitutively phosphorylated osteopontin peptide with anti-calcification activity.

11. The method of claim 10, wherein the administering comprises providing directly to at least one cell of the subject, the constitutively phosphorylated osteopontin peptide in the form of an isolated nucleic acid encoding the constitutively phosphorylated osteopontin peptide operably linked to a promoter.

12. The method of claim 11, wherein the isolated nucleic acid encodes a mutant osteopontin peptide.

13. The method of claim 10, wherein the administering comprises injection of a tissue with the isolated nucleic acid encoding the constitutively phosphorylated osteopontin peptide operably linked to a promoter.

14. The method of claim 10, wherein said administering comprises electroporating a tissue with the isolated nucleic acid encoding the constitutively phosphorylated osteopontin peptide operably linked to a promoter.

15. A method for preventing or retarding the rate of tissue calcification in a device implanted in a subject, the method comprising:
incorporating into at least a portion of the device, prior to insertion of the device into the subject, an osteopontin peptide with anti-calcification activity.

16. The method of claim 15, wherein the therapeutic device is selected from the group consisting of artificial heart valves, and vascular stents.
